(19) United States
(12) Patent Application Publication
(10) Pub. No.: US 2011/0150993 A1
(43) Pub. Date: Jun. 23, 2011

(54) Fine Particle Croscarmellose and Uses Thereof

(75) Inventor: Yeli Zhang, Princeton, NJ (US)

(73) Assignee: FMC CORPORATION, Philadelphia, PA (US)

(21) Appl. No.: 12/970,153

(22) Filed: Dec. 16, 2010

Related U.S. Application Data
(60) Provisional application No. 61/289,048, filed on Dec. 22, 2009.

(51) Int. Cl. A61K 9/10; A61K 9/00; A61K 9/20; A61K 9/14

(52) U.S. Cl. 424/464; 424/400; 424/484; 424/488; 424/487; 424/486; 424/485; 428/402

(57) ABSTRACT

The disclosure is directed to fine particle croscarmellose and its use in various compositions such as solid dosage forms. More specifically, the present disclosure relates to fine particle croscarmellose having a median particle size of 5 μm to 36 μm and a volume mean diameter of 40 μm or less. The specific surface area is typically 0.3 m²/g or more. The fine particle croscarmellose is useful as a disintegrant.

### FIELD OF THE DISCLOSURE

The present disclosure relates to fine particle croscarmellose and its use in various compositions such as solid dosage forms.

### BACKGROUND

Disintegrants are used to aid in the rapid break-up of material and are commonly used in solid dosage forms. Solid dosage forms have a variety of important applications including food and drinks (e.g, confectionery products, aromas, and sweeteners), detergents, dyes, sanitary products (e.g., laundry detergents and other cleaning products), agricultural products, pharmaceuticals, nutraceuticals, etc. Disintegrants assist in the rapid break-up of these solid dosage forms so that their content is quickly released into a target media.

There are a number of factors that affect disintegration. Swelling is believed to be a mechanism in which certain disintegrating agents (such as starch) impart the disintegrating effect. Wicking is believed to another mechanism in which disintegrant agents impart their disintegrating action through porosity and capillary action. Porosity provides pathways for the penetration of fluid.

The manufacturing process (e.g., wet granulation vs. dry granulation vs. direct compression) is another factor which can affect dissolution. For example, in a pharmaceutical application, an active pharmaceutical or nutraceutical ingredient (“API”) can be blended with a variety of excipients, subsequently lubricated and directly compressed into a tablet. A disintegrant used in this type of formulation must simply break the tablet apart to expose the API for dissolution. In a wet granulation process, the API is combined with other excipients and processed with the use of a solvent (aqueous or organic) with subsequent drying and milling to produce granules. The resulting granules are then blended with additional excipients prior to being compressed into a tablet. Dry compaction is similar except that compression and milling are used rather than solvents to make the granules.

Because of the increased demands for faster dissolution, there are now available “superdisintegrants” in addition to disintegrants such as microcrystalline cellulose, starch, pregelatinized starch, and sodium bicarbonate (in combination with citric or tartaric acids). Three major groups of superdisintegrants have been developed which disintegrate in water or aqueous fluid while producing minimal viscosity effects: (1) cross-linked modified starches, (2) cross-linked polyvinylpyrrolidone, and (3) cross-linked carboxymethyl cellulose.

Sodium carboxymethyl starch, also known as sodium starch glycolate (e.g. Explotab®, Primogel®) is cross-linked carboxymethyl potato starch and is believed to act as a super-disintegrant by swelling. At high concentrations, sodium carboxymethyl starch can actually increase disintegration times due to gelling.

Cross-linked polyvinylpyrrolidone, also known as crospovidone (e.g. Polyplasdone XL®, Kollidon CL®) is water insoluble and hydrophilic. It is believed to act as a super-disintegrant primarily by swelling.

Internally cross-linked sodium carboxymethyl cellulose, also known as croscarmellose (e.g. Ac-Di-Sol®, Nymcel®) accelerates disintegration by wicking, swelling, and some deformation recovery due to its fibrous structure.

### SUMMARY OF THE INVENTION

The present disclosure relates to fine particle croscarmellose. It is also directed to the use of fine particle croscarmellose as a disintegrant in various compositions such as solid dosage forms.

More specifically, the present disclosure relates to fine particle croscarmellose having a median particle size (D50) of 5 μm to 36 μm and a volume mean diameter (D[4,3]) of 40 μm or less. The specific surface area is typically 0.3 m²/g or more. In one aspect, the fine particle croscarmellose has a volume mean diameter (D[4,3]) of 35 μm or less. The fine particle croscarmellose typically has a 10^{th} percentile particle size (D10) of 15 μm or less and/or a 90^{th} percentile particle size (D90) of 80 μm or less.

In another embodiment, the present disclosure relates to a fine particle croscarmellose having a median particles size (D50) of 5 μm to less than 25 μm. The specific surface area is typically 0.3 m²/g or more. In one aspect, the fine particle croscarmellose has a volume mean diameter (D[4,3]) of 35 μm or less. The fine particle croscarmellose typically has a 10^{th} percentile particle size (D10) of 15 μm or less and/or a 90^{th} percentile particle size (D90) of 80 μm or less.

In one embodiment, the fine particle croscarmellose is sodium croscarmellose.

In another embodiment, the fine particle croscarmellose is incorporated into a composition.

In yet another embodiment, the fine particle croscarmellose is incorporated into a solid dosage form. The solid dosage form can be any solid dosage form known in the art. For instance, the solid dosage form can be a food or added to a liquid to generate a drink. The solid dosage form can function to deliver aromas, flavors, and sweeteners. The solid dosage form can be detergent, dye, or other sanitary or cleaning product. The solid dosage form can contain an insecticide, a herbicide, or a fungicide. The solid dosage form can be pharmaceutical or nutraceutical.

In one aspect, the solid dosage form is a tablet, a caplet, a capsule (including those made from hard or soft materials such as natural or synthetic gelatin substitutes), a lozenge, a granule, a fine granule, a pill, etc. In another aspect the solid dosage form is an orally disintegrating tablet (ODT). In another aspect the solid dosage form is a tablet that disintegrates in the stomach, the intestines, or other part of the body. In one aspect, the fine particle croscarmellose exists in only part of a solid dosage form. For example, the fine particle croscarmellose can be included in a coating, in a layer, or as part of an internal composition to provide the controlled release of certain contents of the solid dosage form.

The fine particle croscarmellose typically comprises from 0.1 to 25% by weight of the solid dosage form based on the total weight of the solid dosage form. In another aspect the fine particle croscarmellose comprises from 0.1 to 10%, from 0.1 to 5%, from 0.1. to 1%, or about 0.5% by weight of the solid dosage form based on the total weight of the solid dosage form. In one aspect, the solid dosage form further comprises an aroma, a dye, a flavoring, a sweetener, a detergent, a dye, a cleaner, a sanitizer, and/or an API.

The solid dosage form can comprise any matrix known in the art, including but not limited to the group consisting of sucrose, lactose, dextrose, erythritol, fructose, isomalt, lactilol, maltilol, maltose, mannitol, sorbitol, starch, polydextrose, xylitol, calcium phosphate such as dibasic calcium phosphate, calcium carbonate, calcium silicate, silicic acid, carboxymethylcellulose, dextrin, ethylcellulose, gelatin, guar gum, hydrogenated vegetable oil, hydroxyethyl cellulose, hydroxypropyl cellulose, hydroxypropyl methylcellulose, low-substituted hydroxypropyl cellulose, microcrystalline cellulose, polyvinyl pyrrolidone, powdered gum Arabic, glucose, magnesium aluminum silicate, maltodextrin, methylcellulose, polymethacrylates, povidone, sodium alginate, and zein, or a combination thereof.

The solid dosage form can further comprise a lubricant.

The solid dosage form can also comprise one or more colorants, sweeteners, fragrances, flavor blockers, or flavor compounds, or a combination thereof.

### BRIEF DESCRIPTION OF THE FIGURES

FIG. 1: FIG. 1 compares the disintegration time (seconds) versus compaction force (kN) for tablets containing 0.5% by weight of fine particle croscarmellose with tablets containing 0.5% by weight of other commercially available disintegrants.

FIG. 2: FIG. 2 compares the disintegration time (seconds) versus compaction force (kN) for tablets containing 1.0% by weight of fine particle croscarmellose with tablets containing 1.0% by weight of other commercially available disintegrants.

FIG. 3: FIG. 3 compares the disintegration time (seconds) versus compaction force (kN) for tablets containing 2.0% by weight of fine particle croscarmellose with tablets containing 2.0% by weight of other commercially available disintegrants.

FIG. 4: FIG. 4 compares the disintegration time (seconds) versus compaction force (kN) for tablets containing 5.0% by weight of fine particle croscarmellose with tablets containing 5.0% by weight of other commercially available disintegrants.

FIG. 5: FIG. 5 compares the disintegration time (seconds) versus compaction force (kN) for tablets containing 8.0% by weight of fine particle croscarmellose with tablets containing 8.0% by weight of other commercially available disintegrants.

FIG. 6: FIG. 6 compares the disintegration time (seconds) versus compaction force (kN) for tablets containing 12.0% by weight of fine particle croscarmellose with tablets containing 12.0% by weight of other commercially available disintegrants.

FIG. 7: FIG. 7 compares the disintegration time (seconds) versus compaction force (kN) for tablets containing 20.0% by weight of fine particle croscarmellose with tablets containing 20.0% by weight of other commercially available disintegrants.

FIG. 8: FIG. 8 overlays the particle size distribution for commercially available precursor croscarmellose (Ac-Di-Sol®) with the particle size distribution for the fine particle croscarmellose obtained in Example 1.

### DETAILED DESCRIPTION

Where the following terms are used in this specification, they are used as defined below.

The terms “comprising,” “having,” and “including” are used in their open, non-limiting sense.

The terms “a” and “the” are understood to encompass the plural as well as the singular.

By the term “about” when referring to a value, is meant specifically that a measurement can be rounded to the value using a standard convention for rounding numbers. For example, “about 1.5” is 1.45 to 1.54.

The term “particle size distribution” refers to the relative percentages by weight or volume of each of the different size fractions of a particulate matter. The particle size distributions for the present application can be measured using laser light diffraction equipment, such as are sold by Malvern Instruments Ltd., Malvern, Worcestershire, United Kingdom (e.g., Malvern Mastersizer® 2000). Other types of equipment are also suitable for particle size distribution determinations. Laser particle size analysis measures particles using the diffraction and diffusion of a laser beam. During the laser diffraction measurement, particles are passed through a focused laser beam. These particles scatter light at an angle that is inversely proportional to their size. The angular intensity of the scattered light is then measured by a series of photosensitive detectors.

The term “D50” is well-known in the art and refers to the median particle size, i.e., the size where 50 volume percent of the particles have sizes less than the value given.

The term “D[4,3]” is well-known in the art and refers to the volume mean diameter.

The term “D[3,2]” is well-known in the art and refers to the surface area mean diameter (or Souter Mean Diameter).

The term “010” is well-known in the art and refers to the 10^{th} percentile particle size, i.e., the particle size where 10 volume percent of the particles have sizes less than the value given.

The term “D90” is well-known in the art and refers to the 90^{th} percentile particle size i.e., the particle size where 90 volume percent of the particles have sizes less than the value given.

The term “solid dosage form” is well-known in the art and refers to any dosage form that is a solid. Typically, the solid is dry. Non-limiting examples of solid dosage forms include tablets, caplets, capsules (including those made from hard or soft materials such as natural or synthetic gelatin substitutes), lozenges, granules, fine granules, pills, etc.

The solid dosage form can be used in a variety of applications. For example, the solid dosage form can be a food or added to a liquid to generate a drink. The solid dosage form can function to deliver aromas, flavors, and sweeteners. The solid dosage form can be detergent, dye, or other sanitary or cleaning product. The solid dosage form can be a pharmaceutical or nutraceutical. In one aspect, the solid dosage form disintegrates in the stomach, the intestines, or other part of the body. In another aspect, the solid dosage form is an ODT. An ODT typically disintegrates or dissolves in the mouth within 60 seconds or less.

In another aspect, the fine particle croscarmellose exists in only part of a solid dosage form. For example, the fine particle croscarmellose can be included in a coating, in a layer, or as part of an internal composition to provide the controlled release of certain contents of the solid dosage form. The fine particle croscarmellose can be used in a coating to mask a bitter or objectionable taste. The solid dosage form can be an enrobed solid form comprising a film enrobing a compacted fill material having at least one API. The disintegrant can be used in the compacted fill material. Other variations of an enrobed solid form include a layered structure of API in coated pellets to control release of the API.

The solid dosage form can be a suspension tablet. A suspension tablet refers to a tablet that readily disintegrates to form a suspension in liquid. Suspension tablets are useful for delivering a predetermined amount of an API in a drinkable form.

As briefly described above, the present disclosure is directed to fine particle croscarmellose. The fine particle croscarmellose can have a median particle size (D50) of less than about 36 μm, 35 μm. 34 μm. 33 μm, 32 μm, 31 μm, 30 μm, 29 μm, 28 μm, 27 μm, 26 μm, or 25 μm. In one aspect, the median particle size has a lower limit of about 5 μm, 6 μm, 7 μm, 8 μm, 9 μm, 10 μm, 11 μm, 12 μm, 13 μm, 14 μm, or 15 μm. In one aspect, the median particle size (D50) is from 5 μm to 36 μm and in another aspect is from 10 μm to 30 μm. In other aspects, the median particle size (D50) can be less than 25 μm, from 5 μm to 25 μm, or from 10 μm to 20 μm, or any combination thereof.

The fine particle croscarmellose can have a volume mean diameter (D[4,3]) of about 45 μm or less. In one aspect the volume mean diameter (D[4,3]) is less than 40.2 μm. In other aspects the volume mean diameter (D[4,3]) has a lower limit of about 5 μm, 10 μm or 15 μm. In yet other aspects, the volume mean diameter (D[4,3]) is from 5 μm to 45 μm, from 10 μm to 45 μm, from 15 μm to 45 μm, from 5 μm to less than 40.2 μm, from 10 μm to less than 40.2 μm, from 15 to less than 40.2 μm, from 5 μm to 40 μm, from 10 μm to 40 μm, from 15 to 40 μm, from 5 μm to 35 μm, from 10 to 35 μm, from 15 μm to 35 μm, or from 20 μm to 30 μm, or any combination thereof.

The fine particle croscarmellose can have a specific surface area of about 0.3 m²/g or more. In one aspect the specific surface area has an upper limit of about 0.8 m²/g. In other aspects, the specific surface area is greater than or equal to 0.35 m²/g, 0.4 m²/g, 0.45 m²/g, 0.5 m²/g, 0.55 m²/g, or 0.6 m²/g. In yet other aspects, the specific surface is from 0.3 m²/g to 0.8 m²/g, from 0.35 to 0.8 m²/g, from 0.4 m²/g to 0.8 m²/g, from 0.3 m²/g to 0.7 m²/g, from 0.35 m²/g to 0.7 m²/g, from 0.4 m²/g to 0.7, from 0.3 m²/g to 0.6 m²/g, from 0.35 m²/g to 0.6 m²/g, from 0.4 m²/g to 0.6 m²/g, from 0.3 m²/g to 0.55 m²/g, from 0.4 m²/g to 0.55 m²/g, or any combination thereof.

The fine particle croscarmellose can have a surface area mean diameter D[3,2] of about 25 μm, 24 μm, 23 μm, 22 μm, 21 μm, 20 μm, 19 μm, 18 μm, 17 μm, 16 μm, 15 μm or less. In some aspects the surface area mean diameter D[3,2] has a lower limit of about 5 μm, 6 μm, 7 μm, 8 μm, 9, μm, 10, μm, 11, μm, 12 μm, 13 μm, 14, μm, or 15 μm. In other aspects, the surface area mean diameter D[3,2] is less than or equal to 20 μm, 18 μm, 15 μm, or 13 μm. In yet other aspects the surface area mean diameter D[3,2] is from 5 μm to 25 μm, from 5 μm to 20 μm, from μm 5 to 15 μm, or from 10 μm to 15 μm, or any combination thereof.

The fine particle croscarmellose can have a 10^{th} percentile particle size (D10) of 15 μm, 14 μm, 13 μm, 12 μm, 11 μm, 10 μm or less. In some aspects the 10^{th} percentile particle size (D10) has a lower limit of 5 μm, 6 μm, 7 μm, 8 μm, 9 μm, or 10 μm. In other aspects, the 10^{th} percentile particle size (D10) is from 5 μm to 15 μm, from 5 μm to 14 μm, from 5 μm to 13 μm, from 5 μm to 12 μm, from 5 μm to 11 μm, from 5 μm to 10 μm, or any combination thereof.

The fine particle croscarmellose typically has a 90^{th} percentile particle size (D90) of less than about 85 μm. In one aspect, the 90^{th} percentile particle size (D90) has a lower limit of 20 μm. In other aspects the 90^{th} percentile particle size (D90) is less than or equal to 80 μm, 75 μm, 70 μm, 65 μm, 60 μm, 55 μm, 50 μm, 45 μm, 40 μm, or 35 μm. In yet other aspects, the 90^{th} percentile particle size (D90) is from 20 μm to 80 μm, from 20 μm to 75 μm, from 20 μm to 70 μm, from 20 μm to 65 μm, from 25 μm to 60 μm, from 25 μm to 55 μm, from 30 μm to 50 μm, from 30 μm to 45 μm, from 30 μm to 40 μm, or from μm to 35 μm, or any combination thereof.

In one embodiment the fine particle croscarmellose has:

a median particle size (D50) of 5 μm to less than 25 μm;

a volume mean diameter (D[4,3]) of 35 μm or less.

a specific surface area of 0.3 m²/g or more;

a surface area mean diameter (D[3,2]) of 20 μm or less;

a 10^{th} percentile particle size (D10) of 15 μm or less; and

a 90^{th} percentile particle size (D90) of 80 μm or less.

The fine particle croscarmellose can be sodium croscarmellose.

In another embodiment of the present disclosure, the fine particle croscarmellose is incorporated into a composition for use in, for example, a solid dosage form.

In some embodiments, the solid dosage form contains fine particle croscarmellose in an amount less than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% by weight based on the total weight of the solid dosage form. In other aspects the solid dosage form contains a lower limit of fine particle croscarmellose in an amount of about 0.1%, 0.2%, 0.3%, 0.4%, or 0.5% by weight based on the total weight of the solid dosage form. In other aspects, the solid dosage form contains fine particle croscarmellose in an amount from 0.1% to 25%, from 0.1% to 20%, from 0.1% to 15%, from 0.1% to 10%, from 0.1% to 9%, from 0.1% to 8%, from 0.1% to 7%, from 0.1% to 6%, from 0.1% to 5%, from 0.1% to 4%, from 0.1% to 3%, from 0.1% to 2%, or from 0.1% to 1% by weight based on the total weight of the solid dosage form.

There are no limitations with respect to the API that can be incorporated into a solid dosage form. Examples of APIs include, but are not limited to: analgesics: acetaminophen, aspirin, naproxen; anti-ulcer drugs: famotidine; antiemetics: ondansetron, granisetron, dolasetron, domperidone, metoclopramide; antihypertensive drugs: enalapril, losartan, candesartan, valsartan, lisinopril, ramipril, doxazosin, terazosin; antihistaminic drugs: loratadine, cetirizine; antipsychotic drugs: risperidone, olanzapine, quetiapine; antidepressants: paroxetine, fluoxetine, mirtazapine; analgesics and anti-inflammatory drugs: piroxicam; antihypercholesterolemic drugs: simvastatin, lovastatin, pravastatin; antimigraine drugs: zolmitriptan, naratriptan, rizatriptan; anti-epileptic drugs: lamotrigine; anti-Parkinson drugs: selegiline, apomorphine; anxiolytic drugs: diazepam, lorazepam, zolpidem; anti-asthma drugs: zafirlukast, montelukast; erection dysfunction agents: sildenafil; both in their free base form and in their acceptable pharmaceutical salts, hydrates, solvates or isomers. An API can also be one or more of alprazolam, prednisilone, zomitriptan, selegiline, baclofen, carbidopa, levodopa, desloratadine, aripiprazole, loratadine, or donepezil.

The solid dosage form typically has a matrix that binds and holds the ingredients together while in the solid form. The matrix may be a water soluble or insoluble material. Non-limiting examples of matrix materials include dextrose, erythritol, fructose, isomalt, lactilol, maltilol, maltose, mannitol, sorbitol, starch such as corn starch, potato starch, wheat starch, rice starch, partial α-starch, modified starch, partially modified starch, pregelatinized starch, partially pregelatinized starch, starch hydrolysate, polydextrose, and xylitol. Spray dried sorbitol and gamma-crystalline sorbitol are useful. Spray-dried mannitol is a particularly useful matrix. The matrix can be a combination of constituents. One combination is spray-dried mannitol and microcrystalline cellulose. The matrix material can comprise calcium phosphate, dibasic calcium phosphate, precipitated calcium carbonate, calcium silicate, light anhydrous silicic acid, carboxymethylcellulose, dextrin, ethylcellulose, gelatin, guar gum, hydrogenated vegetable oil, hydroxyethyl cellulose, hydroxypropyl cellulose, hydroxypropyl methylcellulose, microcrystalline cellulose, polyvinyl pyrrolidone, powdered gum Arabic, glucose, magnesium aluminum silicate, maltodextrin, methylcellulose, polymethacrylates, sodium alginate, and zein. Moreover, the matrix can have functions in addition to binding. For example, the matrix can provide a sweet or refreshing taste.

Spray dried mannitol is an excellent matrix and is commercially available as Pearlitol® SD (Roquette) and Mannogen™ EZ spray dried mannitol (SPI Pharma). Spray dried mannitol has several useful physical and chemical properties. For example, it dissolves easily in water (1 in 5.5 parts at 20° C.) and quickly (5 g dissolves in approximately 5 sec in 150 mL of water at 20° C.). Direct compression mannitol, powder mannitol, and other related saccharide excipients are slower to dissolve. Spray dried mannitol is substantially in the α crystalline form, whereas other forms of mannitol are generally in the β form. Moreover, spray dried mannitol has flowability of 6 seconds, which is desirable for direct compression processes. It is highly compressible, having a Cohesion Index of 1500-2000. It also has good dilution capacity due to the size and form of the particle, which makes it possible to accept large amounts of API that are not easily compressed. Spray dried mannitol is very chemically stable, is non-hygroscopic, and does not form Maillard reactions with amino groups. Moreover, consumers experience a sense of freshness when taking mannitol because of its negative heat of dissolution. Spray dried mannitol has about half the sweetness of sucrose. It is also very palatability because of its small particle size.

The solid dosage form can contain additives. Non-limiting examples include excipients, additional disintegrants, binders, acidulants, foaming agents, natural and artificial sweeteners, flavoring agents, lubricants, coloring agents, stabilizers, pH control agents, surfactants, etc.

Non-limiting examples of lubricants include magnesium stearate, stearic acid, talc, sodium stearyl fumarate, sucrose fatty acid ester, polyethylenglycol, and waxes. Stearic acid and polyethylene glycol (M_{R}>2000) are known, relatively hydrophilic, lubricants.

Non-limiting examples of additional disintegrants include carboxymethylcellulose, calcium carboxymethylcellulose, sodium carboxymethyl starch, croscarmellose sodium, crospovidone, low-substituted hydroxypropyl cellulose, and hydroxypropyl starch.

Non-limiting examples of acidulants include citric acid, tartaric acid, malic acid, and ascorbic acid.

Non-limiting examples of the foaming agent include sodium hydrogen carbonate, and sodium carbonate.

Non-limiting examples of sweeteners include aspartame, sodium cyclamate, sodium saccharine, ammonium glycyrrhizinate, neohesperidine dihydrochalcone, alitame, neotame, sucralose, stevioside, sucrose, fructose, lactose, sorbitol, and xylitol.

Non-limiting examples of flavoring agents include flavors like menthol, mint, or fruit. Flavors such as raspberry, blackberry, cherry, black cherry, black currant, strawberry, grape, lingonberry, cantaloupe, watermelon, pear, apple, pineapple, mango, peach, apricot, plum, orange, lemon, lime, spearmint, peppermint, vanilla, and chocolate are suitable. Other flavors can include the flavor of bubblegum. The flavor compound can encompass a flavor enhancer, e.g. citric acid.

Non-limiting examples of coloring agents include food colors such as food yellow No. 5, food red No. 2 and food blue No. 2, edible lake pigments, and iron sesquioxide. Furthermore, the colorants can include pigments, natural food colors and dyes suitable for food, drug and cosmetic applications. A full recitation of all F.D. & C. colorants and their corresponding chemical structures can be found in the Kirk-Othmer Encyclopedia of Chemical Technology, 3rd Edition, in volume 5 at pages 857-884, of which text is incorporated herein by reference.

Non-limiting examples of stabilizers include disodium edetate, tocopherol, and cyclodextrin.

Non-limiting examples of pH control agents include citrate, phosphate, carbonate, tartarate, fumarate, acetate, and salts formed with an amino acid.

Non-limiting examples of surfactants include sodium laurylsulfate, polysorbate 80, polyoxyethylene(160), and polyoxypropylene(30)glycol.

### Methods for Preparing Fine Particle Croscarmellose

Fine particle croscarmellose can be prepared by any method known in the art for particle size reduction. For example, fine particle croscarmellose can be prepared by sieving commercially available croscarmellose (e.g., Ac-Di-Sol®) through a mesh sieve. Alternatively, fine particle croscarmellose can be prepared using size-reducing equipment that relies on compression or impact. Compression is applied via moving jaws, rollers or gyratory cones. Impact-based equipment commonly uses hammers or media.

Rolls or impact mills can be used to produce fine particle croscarmellose. Impact mills use revolving hammers to strike incoming particles and to break or fling them against the machine case. The hammers might be fixed or, more commonly, pivoted. Typically, the hammers can be reversed to provide added life before they need to be replaced.

Jet mills can also be used to produce fine particle croscarmellose. In jet mills, particles strike each other as they are transported in a stream of air. For the initial reduction of large materials, a rotating drum propels the feed into the air where the pieces strike each other and fracture.

Ball, pebble and rod mills are also options for producing fine particle croscarmellose. Ball, pebble, and rodmills are rotating cylinders that are partially filled with metal or ceramic balls, flint pebbles or rods. The crushing mechanism is a combination of impact with the grinding media and shearing between the media and the cylinder walls. A variation is a jar mill, in which relatively small ceramic containers holding some grinding media are rotated on a common machine frame.

Some form of separation can follow size reduction. The most common for of separation is simple screening, in which the screen openings are selected to pass the desired size range and retain material that is too large. Screens are subject to blinding by particles large enough to enter a hole, but not able to pass through. Most screens are moved by vibration or regular motion to facilitate passage and to remove overs. Air aspiration is often used, especially in jet mills, to remove fine particles by entrainment while retaining larger particles. Hammer, ball and rod mills frequently have screens on their discharge to retain large particles and media while passing fine particles. Centrifuges or hydroclones, which rely on differences in density and particle size, are also used to separate materials after size reduction.

The following examples illustrate embodiments of the invention but do not limit its scope.

### EXAMPLES

### Example 1

### Preparation of Fine Particle Croscarmellose

Fine particle croscarmellose was prepared by sieving commercially available Ac-Di-Sol® through a 500 mesh sieve. The particle size distribution was determined by analyzing the dry powder using a Malvern Particle Size Analyzer (Mastersizer® 2000, Version 5.54, Malvern Instruments Ltd., Malvern, UK). The fine particle croscarmellose had the particle size distribution shown in the table below.

| | | | | | | Specific |
|---|---|---|---|---|---|---|
| | D10 | D50 | D90 | D[4, 3] | D[3, 2] | Surface Area |
| | (μm) | (μm) | (μm) | (μm) | (μm) | (m²/g) |
| Fine Particle | 10.0 | 26.1 | 66.5 | 33.8 | 14.28 | 0.42 |
| Croscarmellose | | | | | | |

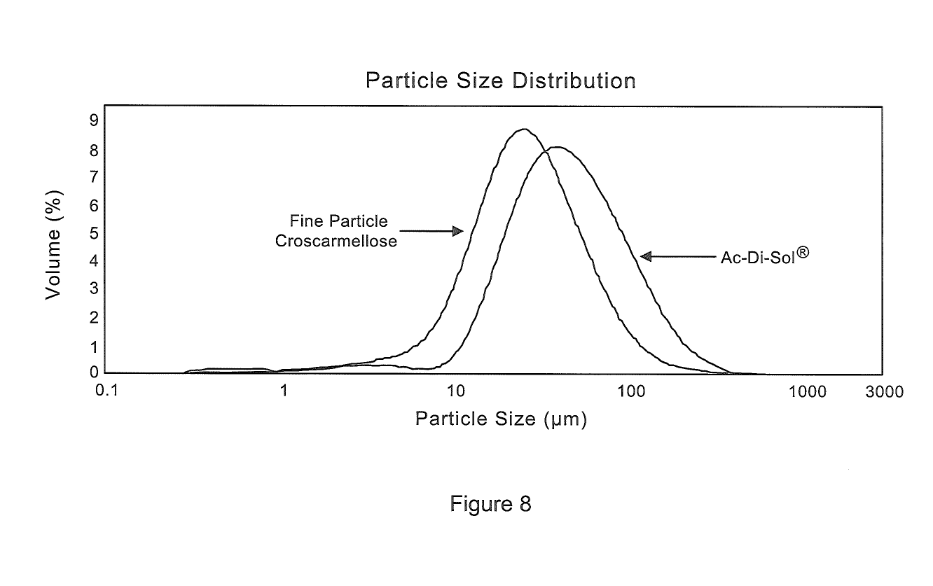

FIG. 8 overlays the particle size distribution for the commercially available precursor Ac-Di-Sol® with the particle size distribution for the fine particle croscarmellose obtained above.

### Example 2

### Comparison of Fine Particle Croscarmellose with Other Disintegrants

Tablets comprising fine particle croscarmellose from Example 1 and commercially available precursor croscarmellose (Ac-Di-Sol®) were prepared and their disintegration time compared with tablets comprising equivalent amounts of other commercially available disintegrants, i.e., crospovidone (PVP® XL-10), crospovidone (Kollidon® CL-SF), and sodium starch glycolate (Glycolys®). The particle size distribution for the various disintegrants including the fine particle croscarmellose is presented in the table below.

Particle Size Distribution of Disintegrants

| | | | | | | Specific |
|---|---|---|---|---|---|---|
| | D10 | D50 | D90 | D[4, 3] | D[3, 2] | Surface Area |
| | (μm) | (μm) | (μm) | (μm) | (μm) | (m²/g) |
| Ac-Di-Sol ® | 17.53 | 44.45 | 114.67 | 57.19 | 27.95 | 0.22 |
| Fine Particle | 10.0 | 26.1 | 66.5 | 33.8 | 14.28 | 0.42 |
| Croscarmellose | | | | | | |
| PVP ® XL-10 | 8.6 | 23.7 | 58.3 | 30.8 | 13.3 | 0.45 |
| Kollidon ® | 2.6 | 7.3 | 26.5 | 18.8 | 3.9 | 1.53 |
| CL-SF | | | | | | |
| Glycolys ® | 22.2 | 41.4 | 73.5 | 45.1 | 36.7 | 0.164 |

Spray-dried mannitol was obtained as Pearlitol® 200 SD from Roquette (Paris, France) which is a direct compressible mannitol, and was used as the tablet matrix. Magnesium stearate (Mallinckrodt, Hazelwood, Mo.) was used as a lubricant. To prepare each formulation, the ingredients were weighed according to the ratios presented in the tables below.

Tablet Formulation Using Ac-Di-Sol ®

| | 0.5% | 1.0% | 2.0% | 5.0% | 8.0% | 12.0% | 20.0% |
|---|---|---|---|---|---|---|---|
| | Tablets | Tablets | Tablets | Tablets | Tablets | Tablets | Tablets |
| Ac-Di Sol ® | 0.5% | 1.0% | 2.0% | 5.0% | 8.0% | 12.0% | 20.0% |
| Mannitol | 98% | 97.5% | 96.5% | 93.5% | 90.5% | 86.5% | 78.5% |
| Magnesium | 1.5% | 1.5% | 1.5% | 1.5% | 1.5% | 1.5% | 1.5% |
| Stearate | | | | | | | |

Tablet Formulation Using Fine Particle Croscarmellose

| | 0.5% | 1.0% | 2.0% | 5.0% | 8.0% | 12.0% | 20.0% |
|---|---|---|---|---|---|---|---|
| | Tablets | Tablets | Tablets | Tablets | Tablets | Tablets | Tablets |
| Fine Particle | 0.5% | 1.0% | 2.0% | 5.0% | 8.0% | 12.0% | 20.0% |
| Croscarmellose | | | | | | | |
| Mannitol | 98% | 97.5% | 96.5% | 93.5% | 90.5% | 86.5% | 78.5% |
| Magnesium | 1.5% | 1.5% | 1.5% | 1.5% | 1.5% | 1.5% | 1.5% |
| Stearate | | | | | | | |

Tablet Formulation Using PVP ® XL-10

| | 0.5% | 1.0% | 2.0% | 5.0% | 8.0% | 12.0% | 20.0% |
|---|---|---|---|---|---|---|---|
| | Tablets | Tablets | Tablets | Tablets | Tablets | Tablets | Tablets |
| PVP ® XL-10 | 0.5% | 1.0% | 2.0% | 5.0% | 8.0% | 12.0% | 20.0% |
| Mannitol | 98% | 97.5% | 96.5% | 93.5% | 90.5% | 86.5% | 78.5% |
| Magnesium | 1.5% | 1.5% | 1.5% | 1.5% | 1.5% | 1.5% | 1.5% |
| Stearate | | | | | | | |

Tablet Formulation Using Kollidon ® CL-SF

| | 0.5% | 1.0% | 2.0% | 5.0% | 8.0% | 12.0% | 20.0% |
|---|---|---|---|---|---|---|---|
| | Tablets | Tablets | Tablets | Tablets | Tablets | Tablets | Tablets |
| Kollidon ® | 0.5% | 1.0% | 2.0% | 5.0% | 8.0% | | |
| CL-SF | | | | | | | |
| Mannitol | 98% | 97.5% | 96.5% | 93.5% | 90.5% | | |
| Magnesium | 1.5% | 1.5% | 1.5% | 1.5% | 1.5% | | |
| Stearate | | | | | | | |

Tablet Formulation Using Glycolys ®

| | 0.5% | 1.0% | 2.0% | 5.0% | 8.0% | 12.0% | 20.0% |
|---|---|---|---|---|---|---|---|
| | Tablets | Tablets | Tablets | Tablets | Tablets | Tablets | Tablets |
| Glycolys ® | 0.5% | 1.0% | 2.0% | 5.0% | 8.0% | 12.0% | 20.0% |
| Mannitol | 98% | 97.5% | 96.5% | 93.5% | 90.5% | 86.5% | 78.5% |
| Magnesium | 1.5% | 1.5% | 1.5% | 1.5% | 1.5% | 1.5% | 1.5% |
| Stearate | | | | | | | |

Each disintegrant and Pearlitol® 200 SD were premixed in a V-blender for 15 minutes; then magnesium stearate was added and followed up with additional 2 minutes of mixing. To prepare tablets, each formulation was compressed individually on a Stokes 512 Tablet Press with four stations. Standard 7/16″ concave punches and corresponding dies were used. Tablet weight was adjusted to 400 mg. SMI Director™ data acquisition system was used to record the compaction process. Compaction forces of 4 kN, 6 kN, 8 kN, 10 kN, or 12 kN were applied to the formulations to produce tablets with different hardness.

Disintegration times of the tablets were determined using a Hanson QC-21 disintegration test system. The test was conducted at 37±0.5 Celsius in a medium of 10 mL distilled water.

Hardness along with tablet weight, thickness, and diameter were determined using an AT4 automatic tablet-testing system (Dr. Schleuniger Pharmatron, Switzerland). The hardness data are reported as the mean hardness. Tablet weight and thickness were controlled in a very tight range.

Tablet friability was measured on a VanKel Friabilator rotated at 25 rpm for 5 minutes. The friability for each sample was calculated using following equation:

Friability (%)=(Wb−Wa)/Wb(100

where Wb and Wa are the weights before and after friability test.

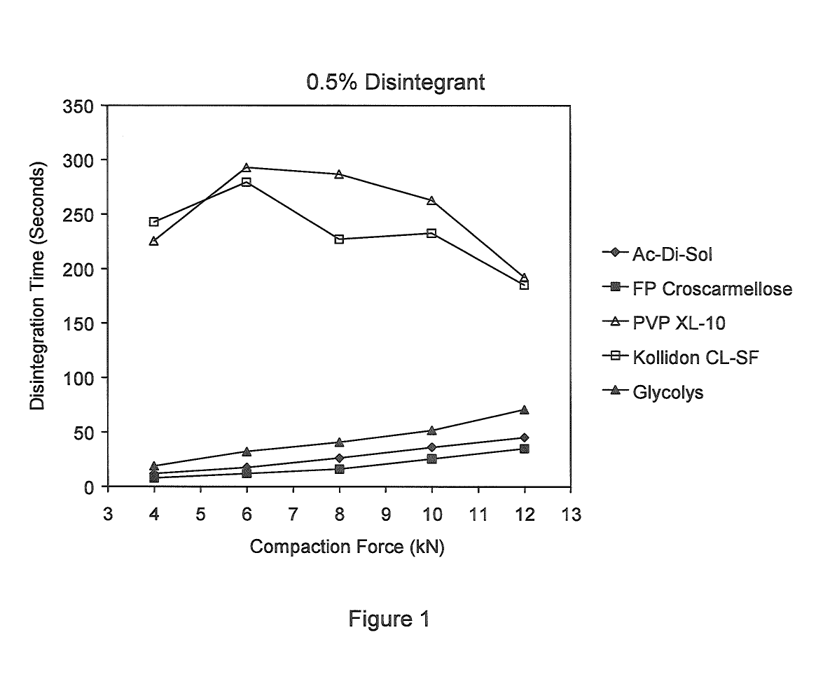

All initial tablet characterization studies (hardness, disintegration time, and friability) were performed on tablets that were stored for 24 hours at ambient condition in closed plastic bags. The data are presented in the tables below and FIGS. 1-7.

0.5% DISINTEGRANT

| | Compaction | | Thick- | | Hard- | Tensile | | |
|---|---|---|---|---|---|---|---|---|
| | Force | Weight | ness | Diameter | ness | Strength | Disintegration | Friability |
| | (kN) | (mg) | (mm) | (mm) | (N) | (Mpa) | (second) | (%) |
| Ac-Di-Sol ® | 4.2 | 402.8 | 5.0 | 11.17 | 26.4 | 0.347 | 11.7 | 0.65 |
| Ac-Di-Sol ® | 6.0 | 402.1 | 4.8 | 11.18 | 40.7 | 0.558 | 17.6 | 0.31 |
| Ac-Di-Sol ® | 8.1 | 402.7 | 4.7 | 11.19 | 61.2 | 0.870 | 26.1 | 0.39 |
| Ac-Di-Sol ® | 10.2 | 403.3 | 4.6 | 11.20 | 81.9 | 1.196 | 36.1 | 0.31 |
| Ac-Di-Sol ® | 12.1 | 403.5 | 4.5 | 11.19 | 97.7 | 1.459 | 45.0 | 0.29 |
| Polyplasdone ® | 4.1 | 397.8 | 5.0 | 10.90 | 23.4 | 0.317 | 123.1 | |
| XL | | | | | | | | |
| Polyplasdone ® | 6.1 | 396.4 | 4.8 | 10.89 | 39.3 | 0.565 | 201.4 | |
| XL | | | | | | | | |
| Polyplasdone ® | 8.1 | 395.4 | 4.6 | 10.90 | 57.5 | 0.846 | 224.5 | |
| XL | | | | | | | | |
| Polyplasdone ® | 10.1 | 397.6 | 4.5 | 10.91 | 74.8 | 1.139 | 186.6 | |
| XL | | | | | | | | |
| Polyplasdone ® | 12.1 | 397.8 | 4.5 | 10.94 | 88.6 | 1.364 | 186.4 | |
| XL | | | | | | | | |
| Polyplasdone ® | 4.0 | 402.1 | 5.1 | 11.14 | 25.7 | 0.333 | 225.3 | 0.69 |
| XL-10 | | | | | | | | |
| Polyplasdone ® | 6.1 | 400.6 | 4.9 | 11.14 | 44.6 | 0.611 | 292.7 | 0.33 |
| XL-10 | | | | | | | | |
| Polyplasdone ® | 8.1 | 402.8 | 4.7 | 11.16 | 65.0 | 0.921 | 286.7 | 0.41 |
| XL-10 | | | | | | | | |
| Polyplasdone ® | 10.1 | 403.0 | 4.6 | 11.15 | 85.2 | 1.237 | 262.6 | 0.32 |
| XL-10 | | | | | | | | |
| Polyplasdone ® | 12.0 | 403.2 | 4.5 | 11.14 | 98.6 | 1.472 | 192.1 | 0.24 |
| XL-10 | | | | | | | | |
| Polyplasdone ® | 4.0 | 400.6 | 5.0 | 10.92 | 23.6 | 0.320 | 217.9 | |
| INF-10 | | | | | | | | |
| Polyplasdone ® | 6.1 | 399.9 | 4.8 | 10.92 | 39.0 | 0.553 | 273.6 | |
| INF-10 | | | | | | | | |
| Polyplasdone ® | 8.2 | 399.5 | 4.7 | 10.91 | 57.4 | 0.843 | 265.6 | |
| INF-10 | | | | | | | | |
| Polyplasdone ® | 10.1 | 398.8 | 4.5 | 10.91 | 78.0 | 1.188 | 221.1 | |
| INF-10 | | | | | | | | |
| Polyplasdone ® | 12.2 | 398.2 | 4.4 | 10.92 | 94.5 | 1.471 | 199.1 | |
| INF-10 | | | | | | | | |
| Kollidon ® CL | 4.2 | 399.2 | 4.9 | 10.89 | 24.3 | 0.337 | 130.6 | |
| Kollidon ® CL | 6.0 | 399.1 | 4.7 | 10.90 | 37.5 | 0.552 | 235.5 | |
| Kollidon ® CL | 8.2 | 401.9 | 4.5 | 10.91 | 64.5 | 0.978 | 232.9 | |
| Kollidon ® CL | 10.0 | 401.0 | 4.5 | 10.90 | 73.6 | 1.120 | 171.1 | |
| Kollidon ® CL | 12.1 | 400.1 | 4.4 | 10.91 | 98.2 | 1.545 | 191.6 | |
| Kollidon ® | 4.1 | 399.8 | 5.0 | 10.90 | 23.7 | 0.319 | 165.0 | |
| CL-F | | | | | | | | |
| Kollidon ® | 6.1 | 401.4 | 4.8 | 10.90 | 39.4 | 0.554 | 262.6 | |
| CL-F | | | | | | | | |
| Kollidon ® | 8.1 | 401.5 | 4.6 | 10.91 | 60.0 | 0.884 | 246.7 | |
| CL-F | | | | | | | | |
| Kollidon ® | 10.2 | 400.3 | 4.5 | 10.91 | 80.2 | 1.225 | 225.0 | |
| CL-F | | | | | | | | |
| Kollidon ® | 12.2 | 402.1 | 4.4 | 10.92 | 97.0 | 1.506 | 187.4 | |
| CL-F | | | | | | | | |
| Kollidon ® | 4.1 | 405.0 | 5.0 | 10.89 | 28.7 | 0.384 | 242.6 | 0.64 |
| CL-SF | | | | | | | | |
| Kollidon ® | 6.0 | 401.9 | 4.8 | 10.90 | 45.0 | 0.639 | 279.2 | 0.41 |
| CL-SF | | | | | | | | |
| Kollidon ® | 8.1 | 402.1 | 4.7 | 10.92 | 66.5 | 0.973 | 226.9 | 0.40 |
| CL-SF | | | | | | | | |
| Kollidon ® | 10.1 | 404.7 | 4.5 | 10.89 | 88.1 | 1.332 | 232.4 | 0.24 |
| CL-SF | | | | | | | | |
| Kollidon ® | 12.1 | 402.2 | 4.5 | 10.90 | 101.3 | 1.561 | 185.1 | 0.25 |
| CL-SF | | | | | | | | |
| Glycolys ® | 4.0 | 401.5 | 5.1 | 11.15 | 27.0 | 0.346 | 18.8 | 0.91 |
| Glycolys ® | 6.0 | 400.3 | 4.9 | 11.15 | 49.7 | 0.679 | 32.1 | 0.53 |
| Glycolys ® | 8.1 | 398.4 | 4.7 | 11.15 | 72.3 | 1.035 | 40.7 | 0.47 |
| Glycolys ® | 10.1 | 401.4 | 4.5 | 11.16 | 103.0 | 1.519 | 51.6 | 0.38 |
| Glycolys ® | 12.1 | 403.8 | 4.5 | 11.17 | 129.6 | 1.937 | 70.8 | 0.27 |
| Fine Particle | 4.0 | 406.7 | 5.1 | 11.00 | 21.6 | 0.280 | 7.6 | 1.17 |
| Croscarmeliose | | | | | | | | |
| Fine Particle | 6.1 | 402.2 | 4.9 | 11.03 | 37.5 | 0.516 | 11.8 | 0.66 |
| Croscarmeliose | | | | | | | | |
| Fine Particle | 8.2 | 400.4 | 4.7 | 11.13 | 55.0 | 0.792 | 16.0 | 0.44 |
| Croscarmeliose | | | | | | | | |
| Fine Particle | 10.2 | 403.0 | 4.5 | 11.07 | 71.3 | 1.068 | 25.5 | 0.32 |
| Croscarmeliose | | | | | | | | |
| Fine Particle | 12.2 | 397.4 | 4.4 | 11.04 | 97.6 | 1.518 | 34.8 | 0.27 |
| Croscarmeliose | | | | | | | | |

Data from the table above for tablets containing 0.5% of disintegrant are shown graphically in FIG. 1.

1% DISINTEGRANT

| | Compaction | | Thick- | | Hard- | Tensile | | |
|---|---|---|---|---|---|---|---|---|
| | Force | Weight | ness | Diameter | ness | Strength | Disintegration | Friability |
| | (kN) | (mg) | (mm) | (mm) | (N) | (Mpa) | (second) | (%) |
| Ac-Di-Sol ® | 4.1 | 397.7 | 5.0 | 11.19 | 24.8 | 0.328 | 11.4 | 0.59 |
| Ac-Di-Sol ® | 6.1 | 397.1 | 4.8 | 11.18 | 41.2 | 0.569 | 13.5 | 0.36 |
| Ac-Di-Sol ® | 8.1 | 398.7 | 4.7 | 11.18 | 61.0 | 0.869 | 18.0 | 0.34 |
| Ac-Di-Sol ® | 10.1 | 402.0 | 4.6 | 11.18 | 76.5 | 1.111 | 26.3 | 0.21 |
| Ac-Di-Sol ® | 12.1 | 401.4 | 4.5 | 11.20 | 95.8 | 1.425 | 31.5 | 0.23 |
| Polyplasdone ® | 4.1 | 399.8 | 5.1 | 10.92 | 20.3 | 0.270 | 20.9 | |
| XL | | | | | | | | |
| Polyplasdone ® | 6.2 | 397.4 | 4.8 | 10.89 | 33.6 | 0.474 | 66.4 | |
| XL | | | | | | | | |
| Polyplasdone ® | 8.2 | 398.0 | 4.6 | 10.93 | 54.6 | 0.804 | 123.2 | |
| XL | | | | | | | | |
| Polyplasdone ® | 10.2 | 398.9 | 4.5 | 10.93 | 74.3 | 1.124 | 138.9 | |
| XL | | | | | | | | |
| Polyplasdone ® | 12.1 | 397.8 | 4.4 | 10.94 | 93.7 | 1.451 | 142.7 | |
| XL | | | | | | | | |
| Polyplasdone ® | 4.0 | 400.9 | 5.1 | 11.16 | 23.2 | 0.300 | 111.4 | 0.88 |
| XL-10 | | | | | | | | |
| Polyplasdone ® | 6.1 | 402.6 | 4.9 | 11.15 | 44.1 | 0.601 | 191.7 | 0.55 |
| XL-10 | | | | | | | | |
| Polyplasdone ® | 8.1 | 401.7 | 4.7 | 11.16 | 62.5 | 0.886 | 248.2 | 0.43 |
| XL-10 | | | | | | | | |
| Polyplasdone ® | 10.1 | 400.9 | 4.6 | 11.15 | 80.6 | 1.178 | 217.5 | 0.28 |
| XL-10 | | | | | | | | |
| Polyplasdone ® | 12.1 | 400.5 | 4.5 | 11.15 | 99.5 | 1.492 | 216.1 | 0.30 |
| XL-10 | | | | | | | | |
| Polyplasdone ® | 4.2 | 400.9 | 5.0 | 10.92 | 24.3 | 0.326 | 158.3 | |
| INF-10 | | | | | | | | |
| Polyplasdone ® | 6.1 | 401.2 | 4.8 | 10.92 | 41.5 | 0.583 | 222.7 | |
| INF-10 | | | | | | | | |
| Polyplasdone ® | 8.1 | 400.0 | 4.7 | 10.94 | 62.0 | 0.899 | 198.1 | |
| INF-10 | | | | | | | | |
| Polyplasdone ® | 10.2 | 399.8 | 4.6 | 10.91 | 82.0 | 1.234 | 191.3 | |
| INF-10 | | | | | | | | |
| Polyplasdone ® | 12.2 | 400.4 | 4.5 | 10.94 | 98.5 | 1.506 | 193.3 | |
| INF-10 | | | | | | | | |
| Kollidon ® CL | 4.1 | 404.8 | 5.1 | 10.91 | 23.8 | 0.315 | 23.6 | |
| Kollidon ® CL | 6.1 | 405.3 | 4.9 | 10.90 | 41.7 | 0.580 | 81.2 | |
| Kollidon ® CL | 8.1 | 401.5 | 4.6 | 10.91 | 60.3 | 0.887 | 140.9 | |
| Kollidon ® CL | 10.2 | 402.3 | 4.5 | 10.91 | 85.6 | 1.294 | 169.8 | |
| Kollidon ® CL | 12.2 | 400.3 | 4.5 | 10.91 | 102.9 | 1.581 | 147.1 | |
| Kollidon ® | 4.1 | 401.3 | 5.0 | 10.91 | 23.3 | 0.312 | 27.8 | |
| CL-F | | | | | | | | |
| Kollidon ® | 6.1 | 401.3 | 4.8 | 10.90 | 40.5 | 0.569 | 78.9 | |
| CL-F | | | | | | | | |
| Kollidon ® | 8.1 | 399.9 | 4.6 | 10.90 | 61.1 | 0.908 | 144.6 | |
| CL-F | | | | | | | | |
| Kollidon ® | 10.2 | 399.4 | 4.5 | 10.90 | 81.6 | 1.235 | 178.5 | |
| CL-F | | | | | | | | |
| Kollidon ® | 12.1 | 401.8 | 4.5 | 10.93 | 103.3 | 1.581 | 175.1 | |
| CL-F | | | | | | | | |
| Kollidon ® | 4.0 | 401.4 | 5.0 | 10.88 | 26.5 | 0.357 | 168.4 | 0.72 |
| CL-SF | | | | | | | | |
| Kollidon ® | 6.1 | 403.1 | 4.8 | 10.88 | 45.9 | 0.646 | 192.3 | 0.43 |
| CL-SF | | | | | | | | |
| Kollidon ® | 8.1 | 403.5 | 4.6 | 10.87 | 68.0 | 1.006 | 221.1 | 0.36 |
| CL-SF | | | | | | | | |
| Kollidon ® | 10.1 | 402.4 | 4.6 | 10.89 | 88.3 | 1.334 | 184.8 | 0.27 |
| CL-SF | | | | | | | | |
| Kollidon ® | 12.2 | 403.3 | 4.5 | 10.91 | 111.7 | 1.720 | 181.7 | 0.29 |
| CL-SF | | | | | | | | |
| Glycolys ® | 4.0 | 402.0 | 5.1 | 11.14 | 27.1 | 0.348 | 17.1 | 1.02 |
| Glycolys ® | 6.0 | 400.8 | 4.9 | 11.15 | 46.5 | 0.635 | 22.4 | 0.52 |
| Glycolys ® | 8.1 | 400.5 | 4.7 | 11.16 | 72.8 | 1.033 | 30.3 | 0.42 |
| Glycolys ® | 10.1 | 399.8 | 4.6 | 11.17 | 95.0 | 1.393 | 40.5 | 0.27 |
| Glycolys ® | 12.1 | 400.4 | 4.5 | 11.16 | 121.4 | 1.823 | 51.1 | 0.29 |
| Fine Particle | 4.0 | 402.2 | 5.0 | 11.00 | 24.4 | 0.323 | 9.0 | 1.18 |
| Croscarmellose | | | | | | | | |
| Fine Particle | 6.1 | 399.9 | 4.9 | 11.00 | 37.6 | 0.515 | 11.4 | 0.65 |
| Croscarmellose | | | | | | | | |
| Fine Particle | 8.2 | 398.8 | 4.6 | 11.01 | 57.3 | 0.841 | 14.7 | 0.50 |
| Croscarmellose | | | | | | | | |
| Fine Particle | 10.1 | 399.5 | 4.5 | 11.12 | 77.5 | 1.166 | 19.3 | 0.29 |
| Croscarmellose | | | | | | | | |
| Fine Particle | 12.1 | 391.4 | 4.4 | 11.16 | 102.6 | 1.569 | 25.7 | 0.26 |
| Croscarmellose | | | | | | | | |

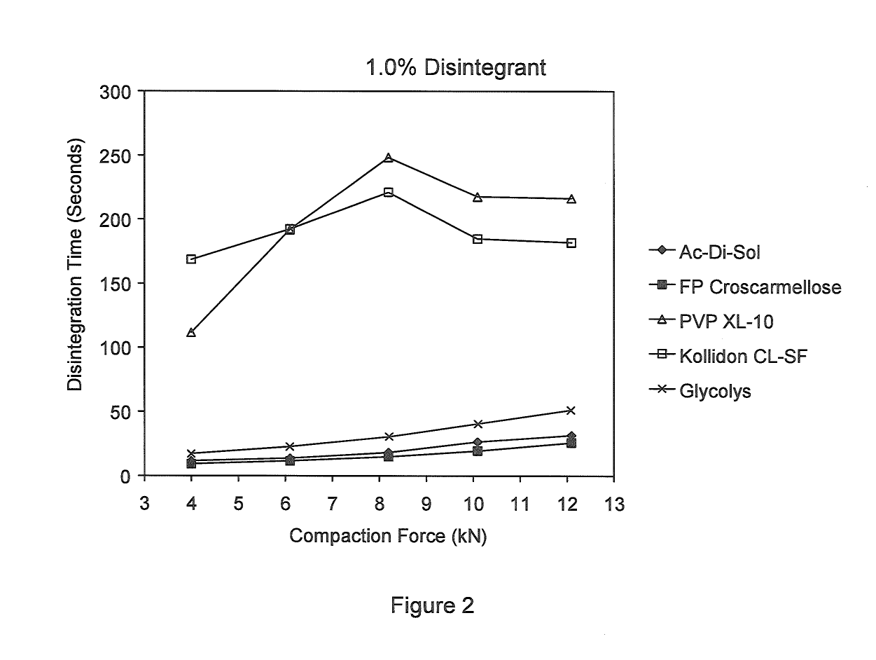

Data from the table above for tablets containing 1.0% of disintegrant are shown graphically in FIG. 2.

2% DISINTEGRANT

| | Compaction | | Thick- | | Hard- | Tensile | | |
|---|---|---|---|---|---|---|---|---|
| | Force | Weight | ness | Diameter | ness | Strength | Disintegration | Friability |
| | (kN) | (mg) | (mm) | (mm) | (N) | (Mpa) | (second) | (%) |
| Ac-Di-Sol ® | 3.9 | 400.9 | 5.1 | 11.17 | 25.1 | 0.322 | 11.7 | 0.85 |
| Ac-Di-Sol ® | 6.1 | 400.6 | 4.9 | 11.17 | 48.1 | 0.656 | 13.6 | 0.50 |
| Ac-Di-Sol ® | 8.2 | 401.4 | 4.7 | 11.18 | 73.6 | 1.041 | 15.0 | 0.33 |
| Ac-Di-Sol ® | 10.2 | 401.1 | 4.6 | 11.17 | 96.6 | 1.403 | 18.1 | 0.29 |
| Ac-Di-Sol ® | 12.2 | 401.6 | 4.5 | 11.18 | 114.7 | 1.708 | 22.9 | 0.23 |
| Polyplasdone ® | 4.0 | 399.9 | 5.1 | 11.15 | 21.4 | 0.276 | 13.0 | 0.97 |
| XL-10 | | | | | | | | |
| Polyplasdone ® | 6.1 | 400.6 | 4.9 | 11.15 | 39.5 | 0.540 | 20.2 | 0.48 |
| XL-10 | | | | | | | | |
| Polyplasdone ® | 8.0 | 404.0 | 4.7 | 11.15 | 60.5 | 0.853 | 24.7 | 0.46 |
| XL-10 | | | | | | | | |
| Polyplasdone ® | 10.0 | 402.4 | 4.6 | 11.16 | 70.6 | 1.024 | 36.3 | 0.37 |
| XL-10 | | | | | | | | |
| Polyplasdone ® | 12.0 | 402.2 | 4.5 | 11.16 | 100.7 | 1.498 | 60.9 | 0.33 |
| XL-10 | | | | | | | | |
| Kollidon ® | 4.0 | 399.1 | 5.1 | 10.90 | 24.4 | 0.322 | 8.8 | 0.83 |
| CL-SF | | | | | | | | |
| Kollidon ® | 6.1 | 397.9 | 4.8 | 10.96 | 45.2 | 0.635 | 12.1 | 0.52 |
| CL-SF | | | | | | | | |
| Kollidon ® | 8.2 | 398.0 | 4.6 | 10.89 | 67.9 | 1.002 | 19.0 | 0.42 |
| CL-SF | | | | | | | | |
| Kollidon ® | 10.2 | 400.0 | 4.6 | 10.91 | 91.6 | 1.377 | 26.8 | 0.30 |
| CL-SF | | | | | | | | |
| Kollidon ® | 12.2 | 399.9 | 4.5 | 10.98 | 113.6 | 1.740 | 46.8 | 0.29 |
| CL-SF | | | | | | | | |
| Glycolys ® | 4.0 | 399.3 | 5.1 | 11.14 | 25.4 | 0.326 | 16.0 | 0.97 |
| Glycolys ® | 6.1 | 401.3 | 4.9 | 11.15 | 47.3 | 0.645 | 20.2 | 0.46 |
| Glycolys ® | 8.1 | 400.5 | 4.7 | 11.16 | 70.7 | 1.005 | 25.6 | 0.43 |
| Glycolys ® | 10.1 | 399.6 | 4.5 | 11.17 | 91.9 | 1.357 | 32.5 | 0.29 |
| Glycolys ® | 12.1 | 401.1 | 4.5 | 11.17 | 117.8 | 1.769 | 39.9 | 0.31 |
| Fine Particle | 4.0 | 398.8 | 5.1 | 11.03 | 18.9 | 0.249 | 11.3 | 1.28 |
| Croscarmellose | | | | | | | | |
| Fine Particle | 6.1 | 401.4 | 4.9 | 11.04 | 38.6 | 0.533 | 13.5 | 0.67 |
| Croscarmellose | | | | | | | | |
| Fine Particle | 8.1 | 401.4 | 4.7 | 11.03 | 60.6 | 0.876 | 14.2 | 0.31 |
| Croscarmellose | | | | | | | | |
| Fine Particle | 10.2 | 398.3 | 4.6 | 11.02 | 76.3 | 1.128 | 15.4 | 0.39 |
| Croscarmellose | | | | | | | | |
| Fine Particle | 12.2 | 404.1 | 4.5 | 11.04 | 104.4 | 1.590 | 18.2 | 0.25 |
| Croscarmellose | | | | | | | | |

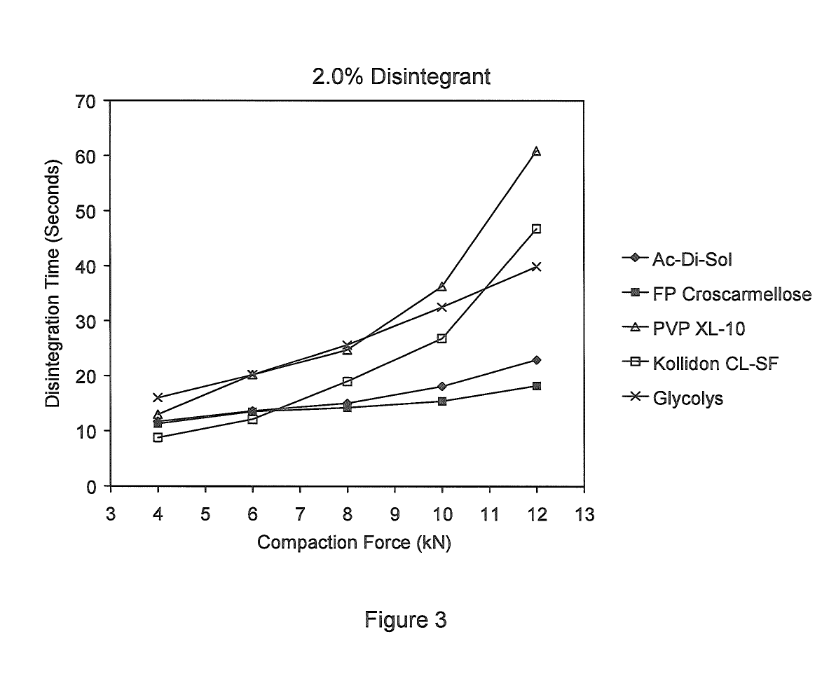

Data from the table above for tablets containing 2.0% of disintegrant are shown graphically in FIG. 3.

5% DISINTEGRANT

| | Compaction | | Thick- | | Hard- | Tensile | | |
|---|---|---|---|---|---|---|---|---|
| | Force | Weight | ness | Diameter | ness | Strength | Disintegration | Friability |
| | (kN) | (mg) | (mm) | (mm) | (N) | (Mpa) | (second) | (%) |
| Ac-Di-Sol ® | 4.1 | 403.0 | 5.1 | 11.18 | 21.4 | 0.274 | 22.0 | 0.89 |
| Ac-Di-Sol ® | 6.1 | 404.9 | 4.9 | 11.18 | 41.7 | 0.564 | 19.3 | 0.39 |
| Ac-Di-Sol ® | 8.1 | 403.1 | 4.7 | 11.18 | 61.0 | 0.860 | 19.0 | 0.31 |
| Ac-Di-Sol ® | 10.2 | 402.4 | 4.6 | 11.19 | 84.6 | 1.229 | 21.0 | 0.27 |
| Ac-Di-Sol ® | 12.2 | 402.8 | 4.5 | 11.18 | 103.7 | 1.544 | 22.0 | 0.21 |
| Polyplasdone ® | 4.1 | 403.4 | 5.2 | 11.20 | 16.0 | 0.201 | 9.3 | 1.08 |
| XL-10 | | | | | | | | |
| Polyplasdone ® | 6.1 | 402.9 | 4.9 | 11.20 | 34.5 | 0.460 | 10.5 | 0.55 |
| XL-10 | | | | | | | | |
| Polyplasdone ® | 8.1 | 403.1 | 4.8 | 11.20 | 52.6 | 0.729 | 13.2 | 0.40 |
| XL-10 | | | | | | | | |
| Polyplasdone ® | 10.2 | 403.5 | 4.7 | 11.19 | 73.8 | 1.055 | 13.6 | 0.25 |
| XL-10 | | | | | | | | |
| Polyplasdone ® | 12.2 | 403.0 | 4.6 | 11.19 | 94.2 | 1.378 | 15.3 | 0.24 |
| XL-10 | | | | | | | | |
| Kollidon ® | 4.0 | 400.2 | 5.2 | 10.91 | 22.5 | 0.291 | 14.6 | 0.92 |
| CL-SF | | | | | | | | |
| Kollidon ® | 6.1 | 400.9 | 4.9 | 10.89 | 48.3 | 0.666 | 15.1 | 0.51 |
| CL-SF | | | | | | | | |
| Kollidon ® | 8.2 | 401.4 | 4.7 | 10.90 | 74.6 | 1.077 | 14.6 | 0.42 |
| CL-SF | | | | | | | | |
| Kollidon ® | 10.1 | 401.5 | 4.6 | 10.90 | 99.6 | 1.479 | 16.3 | 0.35 |
| CL-SF | | | | | | | | |
| Kollidonv ® | 12.1 | 399.9 | 4.5 | 10.96 | 122.9 | 1.858 | 16.7 | 0.29 |
| CL-SF | | | | | | | | |
| Glycolys ® | 4.1 | 400.1 | 5.1 | 11.15 | 24.0 | 0.309 | 21.3 | 1.05 |
| Glycolys ® | 6.0 | 404.3 | 4.9 | 11.15 | 43.8 | 0.595 | 23.4 | 0.54 |
| Glycolys ® | 8.0 | 402.4 | 4.7 | 11.17 | 65.3 | 0.922 | 25.3 | 0.39 |
| Glycolys ® | 10.1 | 401.2 | 4.6 | 11.17 | 90.2 | 1.329 | 31.1 | 0.24 |
| Glycolys ® | 12.0 | 400.8 | 4.5 | 11.16 | 108.4 | 1.630 | 36.0 | 0.27 |
| Fine Particle | 4.0 | 399.4 | 5.0 | 11.19 | 19.2 | 0.252 | 29.0 | 1.59 |
| Croscarmellose | | | | | | | | |
| Fine Particle | 6.1 | 399.0 | 4.8 | 11.19 | 38.6 | 0.539 | 29.5 | 0.73 |
| Croscarmellose | | | | | | | | |
| Fine Particle | 8.2 | 398.7 | 4.6 | 11.19 | 58.2 | 0.842 | 33.1 | 0.44 |
| Croscarmellose | | | | | | | | |
| Fine Particle | 10.1 | 399.9 | 4.5 | 11.20 | 84.3 | 1.256 | 38.2 | 0.32 |
| Croscarmellose | | | | | | | | |
| Fine Particle | 12.1 | 400.6 | 4.4 | 11.19 | 104.5 | 1.601 | 40.4 | 0.26 |
| Croscarmellose | | | | | | | | |

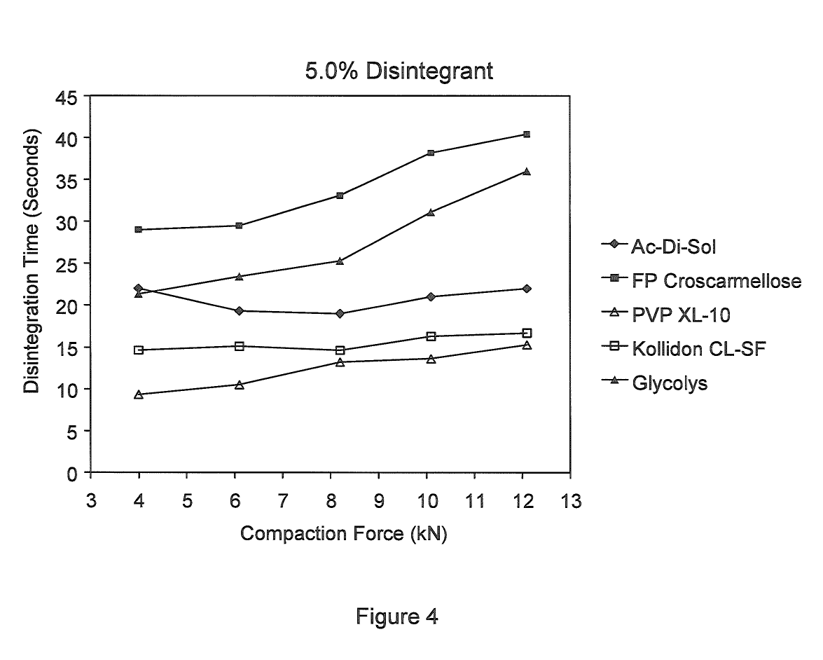

Data from the table above for tablets containing 5.0% of disintegrant are shown graphically in FIG. 4.

8% DISINTEGRANT

| | Compaction | | Thick- | | Hard- | Tensile | | |
|---|---|---|---|---|---|---|---|---|
| | Force | Weight | ness | Diameter | ness | Strength | Disintegration | Friability |
| | (kN) | (mg) | (mm) | (mm) | (N) | (Mpa) | (second) | (%) |
| Ac-Di-Sol ® | 4.1 | 400.6 | 5.1 | 11.21 | 16.2 | 0.208 | 29.8 | 1.05 |
| Ac-Di-Sol ® | 6.1 | 401.1 | 4.9 | 11.19 | 34.8 | 0.473 | 29.5 | 0.48 |
| Ac-Di-Sol ® | 8.2 | 401.3 | 4.7 | 11.18 | 53.8 | 0.763 | 29.1 | 0.34 |
| Ac-Di-Sol ® | 10.2 | 402.4 | 4.6 | 11.18 | 75.6 | 1.101 | 31.2 | 0.29 |
| Ac-Di-Sol ® | 12.2 | 402.4 | 4.5 | 11.18 | 95.0 | 1.414 | 34.5 | 0.22 |
| Polyplasdone ® | 4.1 | 400.0 | 5.2 | 11.22 | 13.7 | 0.170 | 11.4 | 1.33 |
| XL-10 | | | | | | | | |
| Polyplasdone ® | 6.1 | 403.0 | 5.0 | 11.21 | 32.7 | 0.428 | 10.5 | 0.49 |
| XL-10 | | | | | | | | |
| Polyplasdone ® | 8.1 | 399.5 | 4.8 | 11.21 | 54.1 | 0.743 | 13.2 | 0.58 |
| XL-10 | | | | | | | | |
| Polyplasdone ® | 10.1 | 403.2 | 4.7 | 11.20 | 77.0 | 1.086 | 13.6 | 0.24 |
| XL-10 | | | | | | | | |
| Polyplasdone ® | 12.1 | 402.7 | 4.6 | 11.20 | 95.1 | 1.374 | 15.3 | 0.24 |
| XL-10 | | | | | | | | |
| Kollidon ® | 4.0 | 399.4 | 5.3 | 10.93 | 20.6 | 0.261 | 23.5 | 1.05 |
| CL-SF | | | | | | | | |
| Kollidon ® | 6.1 | 399.2 | 5.0 | 11.02 | 43.4 | 0.585 | 21.4 | 0.51 |
| CL-SF | | | | | | | | |
| Kollidon ® | 8.1 | 398.8 | 4.8 | 10.90 | 69.4 | 0.978 | 20.2 | 0.54 |
| CL-SF | | | | | | | | |
| Kollidon ® | 10.2 | 400.9 | 4.7 | 10.92 | 99.1 | 1.453 | 20.4 | 0.39 |
| CL-SF | | | | | | | | |
| Kollidon ® | 12.2 | 401.3 | 4.6 | 10.93 | 125.1 | 1.869 | 19.9 | 0.35 |
| CL-SF | | | | | | | | |
| Glycolys ® | 4.0 | 400.6 | 5.1 | 11.15 | 20.1 | 0.259 | 25.2 | 1.48 |
| Glycolys ® | 6.1 | 401.8 | 4.9 | 11.16 | 41.0 | 0.561 | 29.5 | 0.65 |
| Glycolys ® | 8.1 | 402.9 | 4.7 | 11.16 | 62.7 | 0.890 | 31.5 | 0.46 |
| Glycolys ® | 10.1 | 403.6 | 4.6 | 11.17 | 83.3 | 1.211 | 36.9 | 0.34 |
| Glycolys ® | 12.1 | 402.2 | 4.5 | 11.16 | 100.9 | 1.508 | 37.7 | 0.28 |
| Fine Particle | 4.0 | 400.5 | 5.3 | 11.10 | 13.5 | 0.168 | 58.6 | 6.00 |
| Croscarmellose | | | | | | | | |
| Fine Particle | 6.1 | 402.4 | 4.9 | 11.21 | 23.7 | 0.319 | 77.1 | 0.85 |
| Croscarmellose | | | | | | | | |
| Fine Particle | 8.1 | 401.2 | 4.7 | 11.21 | 39.7 | 0.556 | 63.4 | 0.48 |
| Croscarmellose | | | | | | | | |
| Fine Particle | 10.2 | 402.9 | 4.6 | 11.21 | 61.0 | 0.880 | 85.2 | 0.28 |
| Croscarmellose | | | | | | | | |
| Fine Particle | 12.2 | 402.9 | 4.5 | 11.20 | 81.3 | 1.209 | 81.0 | 1.18 |
| Croscarmellose | | | | | | | | |

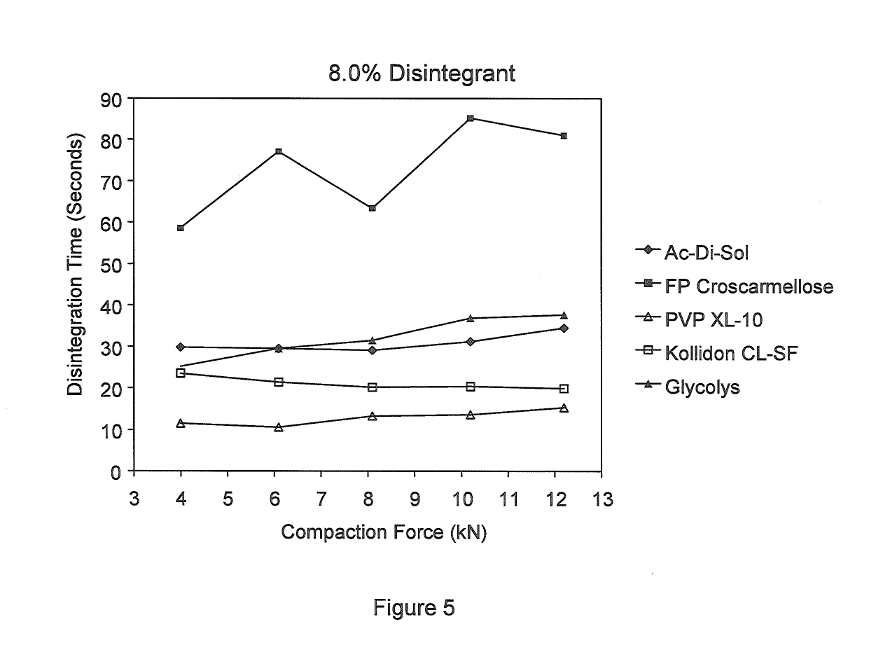

Data from the table above for tablets containing 8.0% of disintegrant are shown graphically in FIG. 5.

12% DISINTEGRANT

| | Compaction | | Thick- | | Hard- | Tensile | | |
|---|---|---|---|---|---|---|---|---|
| | Force | Weight | ness | Diameter | ness | Strength | Disintegration | Friability |
| | (kN) | (mg) | (mm) | (mm) | (N) | (Mpa) | (second) | (%) |
| Ac-Di-Sol ® | 4.0 | | | | | | | 8.55 |
| Ac-Di-Sol ® | 6.1 | 400.0 | 4.9 | 10.97 | 19.3 | 0.265 | 39.8 | 0.97 |
| Ac-Di-Sol ® | 8.2 | 401.7 | 4.7 | 10.94 | 36.6 | 0.524 | 43.3 | 0.47 |
| Ac-Di-Sol ® | 10.2 | 400.5 | 4.6 | 10.94 | 54.0 | 0.799 | 41.8 | 0.32 |
| Ac-Di-Sol ® | 12.2 | 399.8 | 4.5 | 10.95 | 71.4 | 1.081 | 48.3 | 0.29 |
| Polyplasdone ® | 4.0 | | | | | | | 2.35 |
| XL-10 | | | | | | | | |
| Polyplasdone ® | 6.1 | 399.4 | 5.1 | 10.98 | 22.5 | 0.293 | 14.1 | 0.76 |
| XL-10 | | | | | | | | |
| Polyplasdone ® | 8.2 | 399.9 | 4.9 | 10.96 | 41.5 | 0.571 | 13.9 | 0.35 |
| XL-10 | | | | | | | | |
| Polyplasdone ® | 10.1 | 400.2 | 4.8 | 10.97 | 63.7 | 0.904 | 13.6 | 0.23 |
| XL-10 | | | | | | | | |
| Polyplasdone ® | 12.1 | 400.5 | 4.7 | 10.98 | 86.2 | 1.246 | 14.9 | 0.17 |
| XL-10 | | | | | | | | |
| Glycolys ® | 4.0 | | | | | | | 1.72 |
| Glycolys ® | 6.1 | 398.9 | 5.1 | 11.01 | 23.3 | 0.308 | 29.7 | 0.76 |
| Glycolys ® | 8.1 | 399.3 | 4.9 | 10.98 | 41.0 | 0.570 | 33.9 | 0.53 |
| Glycolys ® | 10.2 | 399.8 | 4.8 | 11.00 | 58.7 | 0.833 | 37.5 | 0.40 |
| Glycolys ® | 12.2 | 401.1 | 4.7 | 10.97 | 83.0 | 1.207 | 42.5 | 0.75 |
| Fine Particle | 4.0 | | | | | | 81.5 | 100.00 |
| Croscarmellose | | | | | | | | |
| Fine Particle | 6.1 | 398.7 | 4.8 | 11.18 | 18.3 | 0.251 | 80.4 | 0.80 |
| Croscarmellose | | | | | | | | |
| Fine Particle | 8.2 | 397.5 | 4.7 | 11.17 | 31.3 | 0.448 | 93.2 | 0.38 |
| Croscarmellose | | | | | | | | |
| Fine Particle | 10.2 | 398.6 | 4.5 | 11.16 | 50.2 | 0.741 | 89.8 | 0.21 |
| Croscarmellose | | | | | | | | |
| Fine Particle | 12.2 | 397.8 | 4.4 | 11.15 | 63.9 | 0.971 | 101.8 | 0.21 |
| Croscarmellose | | | | | | | | |

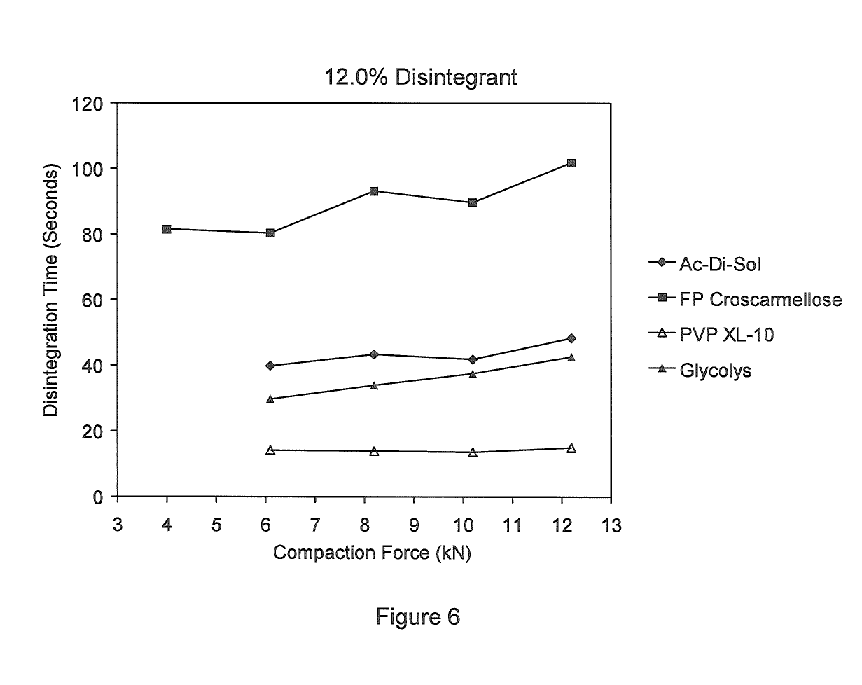

Data from the table above for tablets containing 12.0% of disintegrant are shown graphically in FIG. 6.

20% DISINTEGRANT

| | Compaction | | Thick- | | Hard- | Tensile | | |
|---|---|---|---|---|---|---|---|---|
| | Force | Weight | ness | Diameter | ness | Strength | Disintegration | Friability |
| | (kN) | (mg) | (mm) | (mm) | (N) | (Mpa) | (second) | (%) |
| Ac-Di-Sol ® | 4.0 | | | | | | | |
| Ac-Di-Sol ® | 6.0 | | | | | | 76.4 | 12.52 |
| Ac-Di-Sol ® | 8.2 | 399.5 | 4.9 | 11.07 | 20.0 | 0.275 | 84.4 | 0.34 |
| Ac-Di-Sol ® | 10.2 | 400.4 | 4.7 | 11.01 | 34.3 | 0.494 | 96.4 | 0.29 |
| Ac-Di-Sol ® | 12.2 | 401.3 | 4.6 | 11.00 | 48.5 | 0.714 | 97.1 | 0.22 |
| Polyplasdone ® | 4.0 | | | | | | 25.2 | 10.85 |
| XL-10 | | | | | | | | |
| Polyplasdone ® | 6.1 | 400.9 | 5.3 | 11.03 | 19.3 | 0.243 | 24.3 | 0.91 |
| XL-10 | | | | | | | | |
| Polyplasdone ® | 8.2 | 401.2 | 5.0 | 11.03 | 36.6 | 0.484 | 25.7 | 0.40 |
| XL-10 | | | | | | | | |
| Polyplasdone ® | 10.1 | 401.4 | 4.9 | 11.01 | 58.5 | 0.805 | 26.5 | 0.21 |
| XL-10 | | | | | | | | |
| Polyplasdone ® | 12.1 | 402.5 | 4.8 | 11.02 | 79.3 | 1.120 | 26.5 | 0.13 |
| XL-10 | | | | | | | | |
| Glycolys ® | 4.0 | | | | | | | |
| Glycolys ® | 6.1 | 401.5 | 4.9 | 11.01 | 16.9 | 0.230 | 52.0 | 3.87 |
| Glycolys ® | 8.1 | 401.0 | 4.8 | 10.97 | 27.7 | 0.389 | 55.5 | 3.55 |
| Glycolys ® | 10.2 | 401.1 | 4.7 | 11.00 | 36.9 | 0.530 | 52.3 | 3.12 |
| Glycolys ® | 12.2 | 397.6 | 4.6 | 11.00 | 33.0 | 0.485 | 56.1 | 0.66 |
| Fine Particle | 4.0 | | | | | | | |
| Croscarmellose | | | | | | | | |
| Fine Particle | 6.0 | | | | | | | |
| Croscarmellose | | | | | | | | |
| Fine Particle | 8.2 | | | | | | 190.0 | 100.00 |
| Croscarmellose | | | | | | | | |
| Fine Particle | 10.1 | 402.3 | 4.6 | 11.23 | 14.7 | 0.210 | 221.9 | 4.76 |
| Croscarmellose | | | | | | | | |
| Fine Particle | 12.1 | 401.2 | 4.6 | 11.21 | 23.7 | 0.344 | 219.9 | 2.89 |
| Croscarmellose | | | | | | | | |

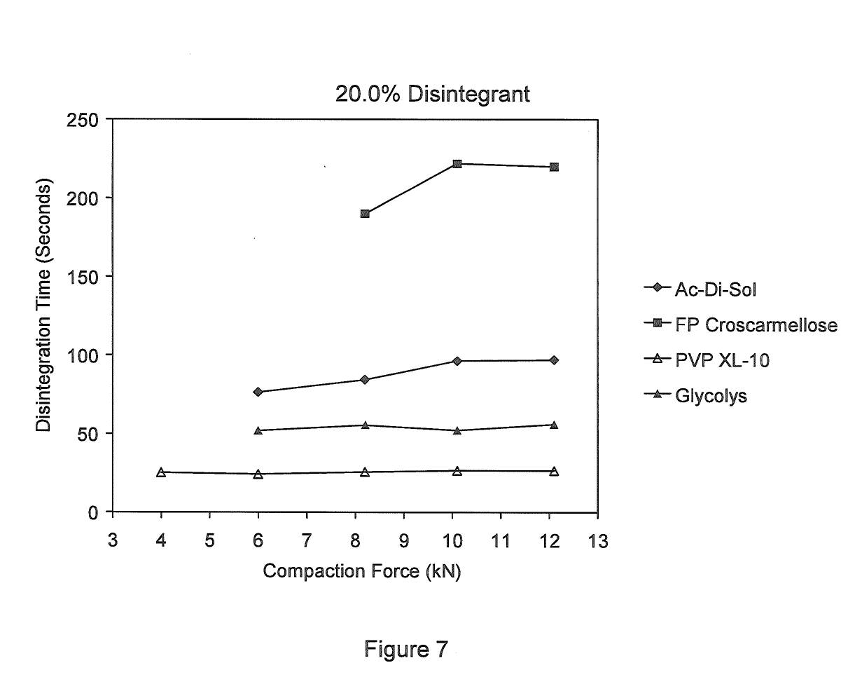

Data from the table above for tablets containing 20.0% of disintegrant are shown graphically in FIG. 7.

Having now fully described the present invention in some detail by way of illustration and example for purposes of clarity of understanding, it will be obvious to one of ordinary skill in the art that the same can be performed by modifying or changing the disclosure within a wide and equivalent range of conditions, formulations and other parameters without affecting the scope of the invention or any specific embodiment thereof, and that such modifications or changes are intended to be encompassed within the scope of the appended claims. All publications, patents, and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

It is claimed:

1. Fine particle croscarmellose having:
a median particle size of 5 μm to 36 μm; and
a volume mean diameter of 40 μm or less.

2. The fine particle croscarmellose according to claim 1 having:
a volume mean diameter of 35 μm or less.

3. The fine particle croscarmellose according to claim 1 having:
a specific surface area of 0.3 m²/g or more.

4. The fine particle croscarmellose according to claim 1 having:
a surface area mean diameter of 20 μm or less.

5. The fine particle croscarmellose according to claim 1 having:
a 10^{th} percentile particle size of 15 μm or less.

6. The fine particle croscarmellose according to claim 1 having:
a 90^{th} percentile particle size of 80 μm or less.

7. The fine particle croscarmellose according to claim 1 having:
a median particle size of 5 μm to less than 25 μm.

8. The fine particle croscarmellose according to claim 7 having:
a volume mean diameter of 35 μm or less.

9. The fine particle croscarmellose according to claim 7 having:
a specific surface area of 0.3 m²/g or more.

10. The fine particle croscarmellose according to claim 7 having:
a surface area mean diameter of 20 μm or less.

11. The fine particle croscarmellose according to claim 7 having:
a 10^{th} percentile particle size of 15 μm or less.

12. The fine particle croscarmellose according to claim 7 having:
a 90^{th} percentile particle size of 80 μm or less.

13. Fine particle croscarmellose having:
a median particle size of 5 μm to less than 25 μm;
a volume mean diameter of 35 μm or less.
a specific surface area of 0.3 m²/g or more;
a surface area mean diameter of 20 μm or less;
a 10^{th} percentile particle size of 15 μm or less; and
a 90^{th} percentile particle size of 80 μm or less.

14. The fine particle croscarmellose according to claim 13, wherein the fine particle croscarmellose is sodium croscarmellose.

15. A composition comprising the fine particle croscarmellose of claim 1.

16. A solid dosage form comprising an active pharmaceutical or nutraceutical ingredient (API) and the fine particle croscarmellose of claim 1, wherein the fine particle croscarmellose is present in an amount from 0.1 to 25% by weight based on the total weight of the solid dosage form.

17. The solid dosage form according to claim 16, wherein the fine particle croscarmellose is present in an amount from 0.1 to 10% by weight based on the total weight of the solid dosage form.

18. The solid dosage form according to claim 17, wherein the fine particle croscarmellose is present in an amount from 0.1 to 5% by weight based on the total weight of the solid dosage form.

19. The solid dosage form according to claim 18, wherein the fine particle croscarmellose is present in an amount from 0.1 to 1% by weight based on the total weight of the solid dosage form.

20. The solid dosage form according to claim 19, wherein the fine particle croscarmellose is present in an amount of about 0.5% by weight based on the total weight of the solid dosage form.

21. The solid dosage form according to claim 16, further comprising a soluble or insoluble matrix.

22. The solid dosage form according to claim 16 further comprising a matrix selected from the group consisting of sucrose, lactose, dextrose, erythritol, fructose, isomalt, lactilol, maltilol, maltose, mannitol, sorbitol, starch, polydextrose, xylitol, calcium phosphate, dibasic calcium phosphate, calcium carbonate, calcium silicate, silicic acid, carboxymethylcellulose, dextrin, ethylcellulose, gelatin, guar gum, hydrogenated vegetable oil, hydroxyethyl cellulose, hydroxypropyl cellulose, hydroxypropyl methylcellulose, low-substituted hydroxypropyl cellulose, microcrystalline cellulose, polyvinyl pyrrolidone, powdered gum Arabic, glucose, magnesium aluminum silicate, maltodextrin, methylcellulose, polymethacrylates, povidone, sodium alginate, and zein, or a combination thereof.

23. The solid dosage form according to claim 16 further comprising a lubricant.

24. The solid dosage form according to claim 16 further comprising one or more colorants, sweeteners, fragrances, flavor blockers, flavor compounds, or additional disintegrants, or a combination thereof.

25. The solid dosage form according to claim 16 that is a tablet.

26. The tablet according to claim 25 that is an orally disintegrating tablet (ODT).

27. The fine particle croscarmellose according to claim 1 produced by attriting or sieving croscarmellose.

28. The fine particle croscarmellose according to claim 27 produced by sieving.

29. The fine particle croscarmellose according to claim 27 produced by milling.
